(19) United States
(12) Plant Patent Application Publication
(10) Pub. No.: US 2026/0182479 P1
(43) Pub. Date: Jun. 25, 2026

(54) Strawberry Plant Named 'CBC022'

(50) ;

(71) Applicants: Kyle M VandenLangenberg, Tracy, CA (US); Todd G Etchandy, Casmarillo, CA (US)

(72) Inventors: Kyle M VandenLangenberg, Tracy, CA (US); Todd G Etchandy, Casmarillo, CA (US)

(73) Assignee: California Berry Cultivars, LLC, French Camp, CA (US)

(21) Appl. No.: 19/427,354

(22) Filed: Dec. 19, 2025

Related U.S. Application Data
(60) Provisional application No. 63/738,142, filed on Dec. 23, 2024.

(51) Int. Cl. A01H 6/74; A01H 5/08

(52) U.S. Cl. CPC A01H 6/7409; A01H 5/08
USPC PLT/208

(57) ABSTRACT

A new and distinct cultivar of strawberry plant (Fragaria x ananassa) named ‘CBC022’ is presented here. This new short-day strawberry cultivar is characterized by moderate vigor plants which produce exceptionally large yields of attractive conical to blocky fruit with high firmness throughout the growing season.

LATIN NAME OF THE GENUS AND SPECIES OF THE PLANT CLAIMED Botanical classification: Fragaria x ananassa.

Variety Denomination: The new strawberry variety denomination is ‘CBC022’.

### BACKGROUND OF THE INVENTION

This invention relates to a new and distinctive short-day type cultivar designated as ‘CBC022’. The new cultivar was the result of a controlled cross performed in 2019 in Oxnard, California between the unpatented selection ‘115.066-601’ and the unpatented selection ‘115.015-606’. ‘CBC022’ was first fruited at an experimental orchard near Oxnard, Calif. in 2019, where it was selected, originally designated ‘119.045-602’, and propagated asexually by runners. Following selection and during testing, the plant of this selection was later designated ‘CBC022’. Asexual propagules from this original source have been tested in Watsonville, Calif., in Oxnard, Calif., and to a limited extent in grower fields starting in 2023. The cultivar is stable and reproduces true to type in successive generations of asexual reproduction.

### Comparison with Parents

The parent ‘115.066-601’ is a short-day selection chosen for its high yield and good appearance. The parent ‘115.015-606’ was chosen for its general combining ability, having large size fruit with good firmness.

### SUMMARY OF THE INVENTION

‘CBC022’ is a short-day cultivar that produces fruit over an extended period when treated appropriately in arid, subtropical climates. When treated with appropriate planting regimes, ‘CBC022’ has larger fruit and produces individual plant yields greater than that of either parent. The following traits have been repeatedly observed over multiple testing years and locations and are determined to be unique characteristics of ‘CBC022’. These characteristics in combination distinguish ‘CBC022’ as a new and distinct Strawberry plant:
1. Medium sized plant with open canopy.
2. Short-day flowering habit.
3. Large glossy red fruit.
4. Fruit is firm throughout the growing season.
5 Fruit postharvest longevity is good.

The cultivar ‘CBC022’, when planted under appropriate conditions, is a photo-period sensitive flowering short-day with exceptionally high total yields. Fruit quality is good with excellent flavor.

### BRIEF DESCRIPTION OF THE FIGS.

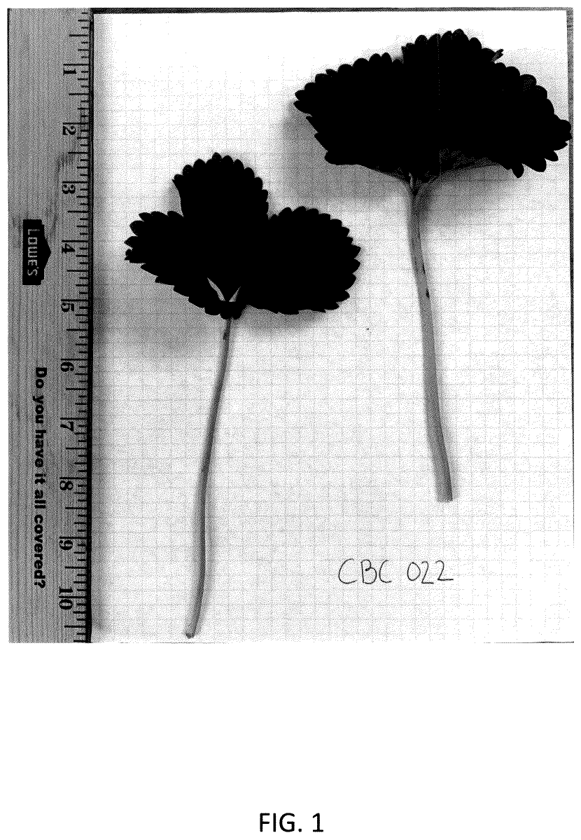

The accompanying color photographs, identified as FIGS. 1 through 6, show the appearance of typical specimens of the new strawberry cultivar designated ‘CBC022’. These Figures depict the colors, as nearly true as it is reasonably possible given differences in color illustrations of this character. Accordingly, color in the photographs may differ slightly from the colors discussed in the botanical description.

FIG. 1 shows a typical leaf and petiole structure.

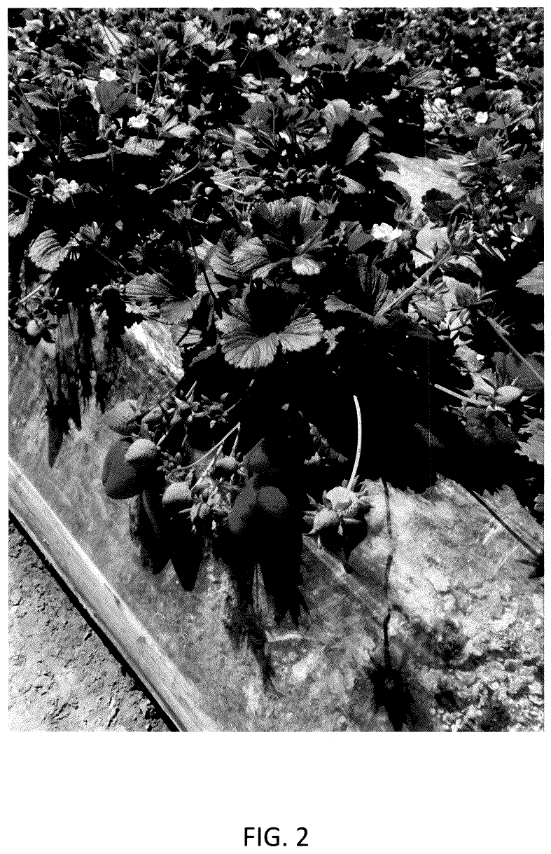

FIG. 2 shows a typical plant and fruit early season.

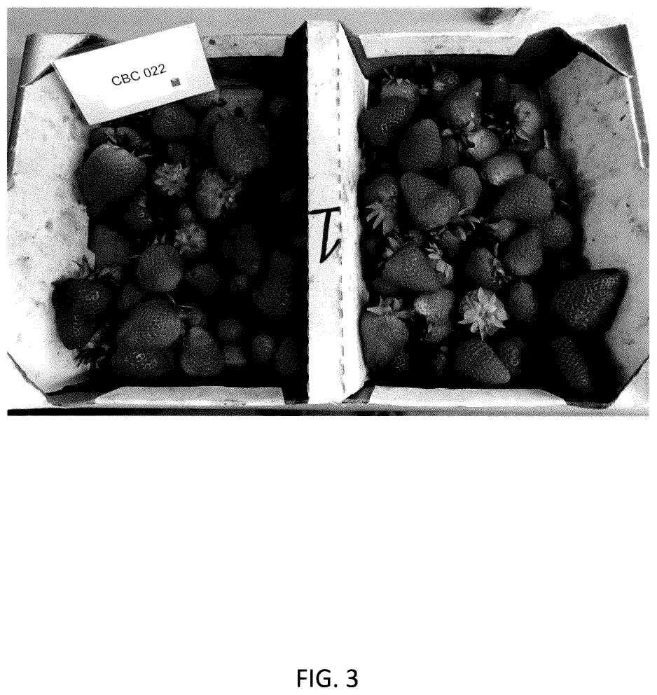

FIG. 3 shows typical fruit in a box.

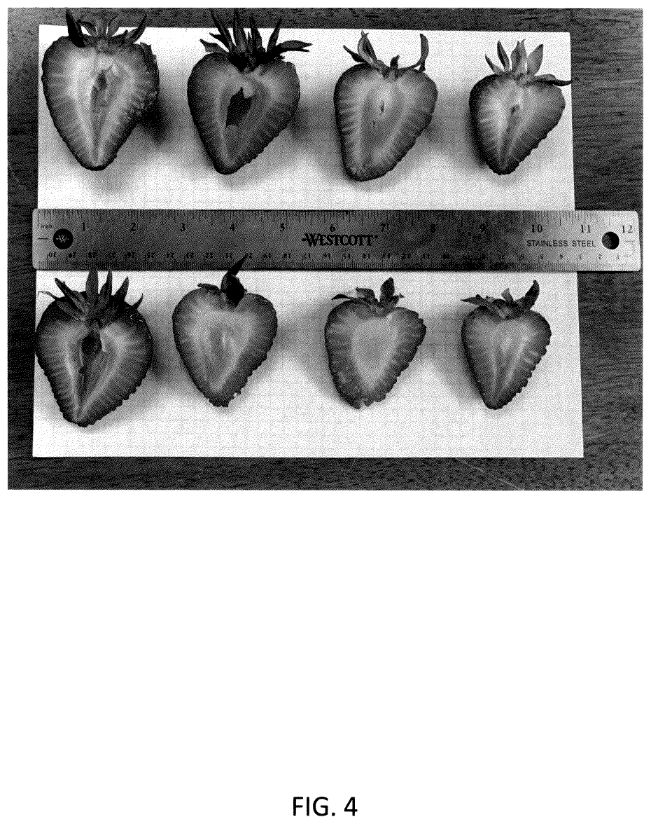

FIG. 4 shows a cross-section of representative fruit mid-season.

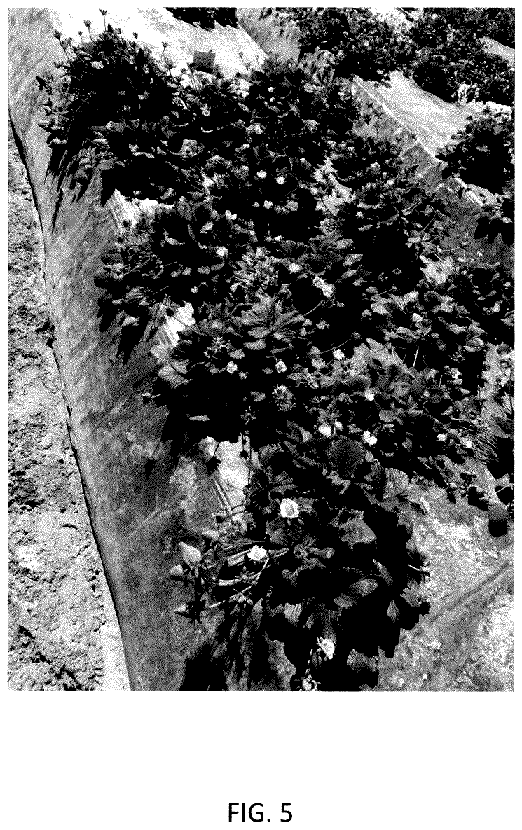

FIG. 5 shows a typical plot in the field of plants and fruit.

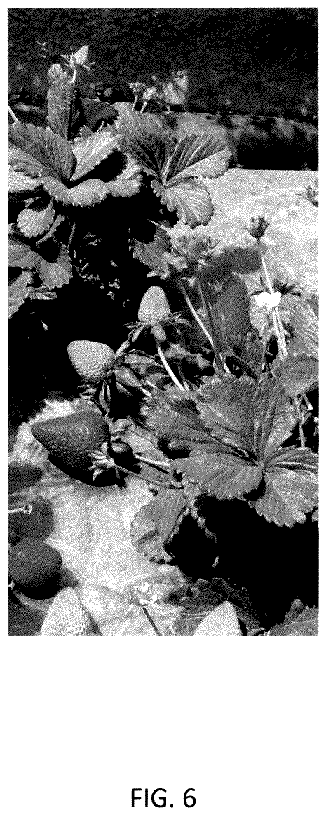

FIG. 6 shows a typical plant and fruit early season with open canopy.

### DETAILED BOTANICAL DESCRIPTION OF THE PLANT

‘CBC022’ is a new and distinct short-day cultivar of strawberry, genus and species Fragaria x ananassa. It is the result of a controlled cross between the selection ‘115.066-601’ and the selection ‘115.015-606’. Plants of ‘CBC022’ bloom under short-day conditions typically encountered in strawberry growing regions in Southern California. The plants of the new cultivar have a moderate vigor. ‘CBC022’ exhibits several characteristics which are improvements over one or both of its parent varieties, and other cultivars known to the Inventors. These characteristics are compared with other cultivars in Table 2, 3, and 4.

The fruit size of ‘CBC022’ is large, with a seasonal average of 44.7 g, larger than its parents. The fruit size of ‘CBC022’ is ideal since it is not too large to be damaged in shipping containers, but not too small to allow for efficient picking and packing. Storability of the fruit has not been measured but in informal observations the fruit retains good physical characteristics and appearance for several days after harvest at room temperature and up to at least a week in common refrigerator conditions of roughly 3° C.

‘CBC022’ produces mostly conic, some blocky shaped fruit. The fruit of ‘CBC022’ generally ripens evenly, resulting in an even external color over the whole fruit. The fruit of ‘CBC022’ is firm throughout the growing season.

The data set for ‘CBC022’ set forth below was collected in May 2025 from plants grown at a test plot in Watsonville, CA. Yield, size, and firmness data is either averaged over the entire 2024-2025 growing season or presented as monthly totals from the Oxnard, CA test plot. Description data was collected on five-month-old plants.

The observed characteristics of ‘CBC022’ are set forth in Table 1, 2, 3, and 4 below. Color terminology is in accordance with the Pantone Color Chart. The observed characteristics of ‘CBC022’ are also compared to cultivars ‘Fronteras’ (U.S. Plant Pat. No. 26,709) and ‘UCD Victor’ (U.S. Plant Pat. No. 28,880), cultivars known to the Inventor, in Table 2, 3, and 4. Table 5 sets forth the test results of DNA fingerprinting using eight SSR markers.

TABLE 1
Detailed Description of Characteristics of ‘CBC022

| | |
|---|---|
| SPECIFICATION: | |
| Genus/Species | Fragaria X ananassa |
| Market Name | Strawberry |
| PARENTS: | |
| Female | ‘115.066-601’ |
| Male | ‘115.015-606’ |
| PLANT: | |
| Type | Short-day, non-everbearing |
| Growth Habit | Upright |
| Foliage density | Moderate |
| Vigor | Moderate |
| Height | Average 19 cm; range 17-22 cm |
| Width | Average 33 cm; range 29-37 cm |
| Disease tolerance | None observed |
| LEAF: | |
| Width | Average 11 cm; range 10-13 cm |
| Color | Adaxial surface: Pantone 2266 C |
| | Abaxial surface: Pantone 4200 C |
| Pubescence | Slight |
| Glossiness | Low |
| Variegation | Not present |
| TERMINAL LEAFLET | |
| Length | Average 66 mm; range 56-77 |
| Width | Average 53 mm; range 45-64 |
| Ratio (length/width) | 1.25 |
| Margins | Crenate to serrate |
| Leaflet shape | Obovate |
| Base shape | Cuneate |
| Cross-section shape | Concave |
| PETIOLE | |
| Pubescence | Medium density |
| Petiole color | Pantone 2303 C |
| Petiole length | Average 14 cm; range 10-18 cm |
| Petiole diameter | Average 3.5 mm; range 2-4 mm |
| STIPULE | |
| Anthocyanin coloration | Not present |
| Length | Average 22 mm; range 16-29 mm |
| Width | Average 9 mm, range 8-10 mm |
| INFLORESCENCE: | |
| Flowering | Short-day |
| Position | Mostly at or above canopy |
| Petal arrangement | Slightly overlapping |
| Calyx size | Larger than corolla |
| Number of blooms | 29 |
| Length | 2 cm |
| Flower diameter | Average 37 mm; range 32-44 mm |
| Petal number | Average 6; range 5-8 |
| Petal length | Average 17 mm; range 15-19 mm |
| Petal width | Average 16 mm; range 15-17 mm |
| Ratio (length/width) | 1.06 |
| Petal color | Pantone 000 C |
| Calyx color | Adaxial surface: Pantone 555 C |
| | Abaxial surface: Pantone 4207 C |
| Stamens length | Average 25 mm; range 20-30 mm |
| PEDICEL | |
| Pubescence | Slight |
| Length | Average 159 mm; range 120-195 mm |
| Width | Average 3 mm; range 2-4 mm |
| Ratio (length/width) | 53 |
| Color | Pantone 2303 C |
| FRUIT: | |
| Bearing | Short-day, non-everbearing |
| Individual fruit weight | Seasonal average: 44.7 g |
| Shape | Mostly conic, some blocky |
| Achenes | Just below fruit surface |
| Glossiness | Medium |
| External color evenness | Even |
| External color | Pantone 1807 C |
| Internal color | Pantone 486 C |
| Band at top without | Narrow width |
| achenes | |
| Fruit Yield | Grams per plant: 1749 g |
| Firmness | 0.56 kg/force |
| Achene color | Pantone 7628 C |

TABLE 2
Marketable yield of ‘CBC022’ compared to other cultivars known
to the Inventors in Oxnard, CA during the 2024-2025 season.

| | Marketable yield (g/plant) | | | | | |
|---|---|---|---|---|---|---|
| Cultivar | Dec. | Jan. | Feb. | Mar. | Apr. | Total |
| ‘CBC022’ | 4 | 109 | 161 | 286 | 1189 | 1749 |
| ‘Fronteras’ | 6 | 100 | 132 | 325 | 870 | 1433 |
| ‘UCD Victor’ | 3 | 42 | 103 | 485 | 935 | 1568 |

TABLE 3
Size of ‘CBC022’ compared to other cultivars known
to the Inventors in Oxnard, CA during the 2024-2025 season.

| | Fruit size (g/fruit) | | | | | |
|---|---|---|---|---|---|---|
| Cultivar | Dec. | Jan. | Feb. | Mar. | Apr. | Average |
| ‘CBC022’ | 50.5 | 43.2 | 43.2 | 47.2 | 39.3 | 44.7 |
| ‘Fronteras’ | 37.9 | 42.6 | 41.6 | 39.3 | 30.7 | 38.4 |
| ‘UCD Victor’ | 57.0 | 38.5 | 38.3 | 44.4 | 30.0 | 41.7 |

TABLE 4
Firmness of ‘CBC022’ compared to other cultivars known
to the Inventors in Oxnard, CA during the 2024-2025 season.
Firmness (kg/force)

| Cultivar | Mar. | Apr. | Average |
|---|---|---|---|
| ‘CBC022’ | 0.59 | 0.52 | 0.56 |
| ‘Fronteras’ | 0.46 | 0.46 | 0.46 |
| ‘UCD Victor’ | 0.58 | 0.46 | 0.52 |

TABLE 5
DNA finger printing results of eight SSR markers.

| | Allelic Fingerprint Analysis | | | | | | | |
|---|---|---|---|---|---|---|---|---|
| Cultivar | M1 | M2 | M3 | M4 | M5 | M6 | M7 | M8 |
| ‘CBC022’ | 206, | 259, | 190, | 279, | 154, | 242, | 121, | 213, |
| | 224, | 269, | 216, | 289, | 156 | 251, | 124, | 222, |
| | 229 | 271 | 232 | 292 | | 257, | 130, | 228 |
| | | | | | | 264 | 138 | |

We claim:

1. A new and distinct strawberry plant named ‘CBC022’ as described and illustrated herein.
